(19) United States
(12) Patent Application Publication
(10) Pub. No.: US 2010/0228120 A1
(43) Pub. Date: Sep. 9, 2010

(54) SYSTEM AND METHOD OF POSITIONING A SENSOR FOR ACQUIRING A VITAL PARAMETER OF A SUBJECT

(75) Inventors: Jeroen Adrianus Johannes Thijs, Aachen (DE); Olaf Such, Aachen (DE); Jens Muehlsteff, Aachen (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/376,319

(22) PCT Filed: Jul. 17, 2007
(86) PCT No.: PCT/IB2007/052841

§ 371 Date: Feb. 4, 2009

(30) Foreign Application Priority Data
Aug. 9, 2006 (EP) 06118637.5

(51) Int. Cl. A61B 5/05

(52) U.S. Cl. 600/426

(57) ABSTRACT

Doppler radar sensor (3) is used for generating a reference or target signal (S_{R}, S_{T}), which can be used for positioning of measuring sensors (2) for acquiring a vital parameter of a subject (5). With the invention a simple and reliable technique for positioning sensors (2) is provided. Furthermore a simple and reliable technique for reproducing the position of sensors (2) is provided.

The present invention relates to a system and method of generating a reference signal for the positioning of a sensor for acquiring a vital parameter of a subject and an according system and method of positioning such a sensor. The present invention further relates to a system and method of generating a target signal for the positioning of a sensor for acquiring a vital parameter of a subject and an according system and method of positioning such a sensor. The present invention further relates to the use of a Doppler radar acquisition device for such methods.

More and more medically related measurements are performed in a non-invasive way. These measurements often involve the positioning of sensors, largely electrodes on a subject's body, e.g. the subject's thorax. Electrocardiogram (ECG) electrodes are well known examples, however, other measurements are also increasingly performed non-invasively. For example impedance cardiography using four or eight ECG electrodes is another common non-invasive measurement for mechanical parameters of the heart. Especially the measurement of body composition and cardiac output using bio-impedance have special requirements to get reproducible measurements.

For most measurements the reproducibility plays an important role. Positioning the sensors for a measurement at the same position is a key element of reproducing a measurement. In case of a standard ECG the positioning of the electrodes is essential to obtain a measurement. Differences in the positioning of the electrodes cause differences in the ECG vector that is observed and can make the difference between a diagnostically relevant and a non-relevant measurement. In case of impedance cardiography the positioning of the electrodes can cause large differences in measurement outcomes. In order to get reproducible measurements, the positioning of the electrodes is an important challenge. In case of heart sound measurements the exact positioning of the sensors is important to obtain a reliable measurement. The quality of a measurement often also depends on the right positioning of the sensors. Getting the right electrode positions e.g. for ECG in order to get the standard twelve leads is a major part of the work flow burden for every cardiologist.

It is an object of the present invention to provide a simple and reliable technique for positioning sensors for acquiring vital parameters of a subject.

This object is achieved according to the invention by a system and method of generating a reference signal and a corresponding system and method of positioning a sensor using said reference signal as well as by a computer program, as described below.

The method of generating a reference signal for the positioning of a sensor for acquiring a vital parameter of a subject, comprises the steps of:
positioning the sensor on the surface of the subject's body,
acquiring by means of a Doppler radar acquisition device a reference body signal representing a movement of an object within the subject's body, the position of the Doppler radar acquisition device being associated in a defined way with the position of the sensor, and
storing the acquired reference body signal by means of a data storage device.

The corresponding method of positioning a sensor for acquiring a vital parameter of a subject comprises the steps of:
during movement of the sensor across the surface of the subject's body, acquiring by means of a Doppler radar acquisition device a current body signal representing a movement of an object within the subject's body, the position of the Doppler radar acquisition device being associated in a defined way with the position of the sensor,
comparing by means of a processing device the current body signal with a reference body signal and
generating by means of the processing device a positioning signal depending on the result of the comparison.

The system for generating a reference signal for the positioning of a sensor for acquiring a vital parameter of a subject comprises:
a Doppler radar acquisition device adapted to acquire a reference body signal representing a movement of an object within the subject's body, the position of the Doppler radar acquisition device being associated in a defined way with the position of the sensor, which is positioned on the surface of the subject's body, and
a data storage device adapted to store the acquired reference body signal.

The corresponding system for acquiring a vital parameter of a subject by means of a sensor using said reference signal, comprises:
a Doppler radar acquisition device adapted to acquire a current body signal during movement of the sensor across the surface of the subject's body, the body signal representing a movement of an object within the subject's body, the position of the Doppler radar acquisition device being associated in a defined way with the position of the sensor,
a processing device adapted to compare the current body signal with a reference body signal, and further adapted to generate a positioning signal depending on the result of the comparison.

The corresponding computer program to be executed in a computer comprises:
computer instructions to compare a current body signal with a reference body signal, said current body signal being acquired by means of a Doppler radar acquisition device during movement of a sensor across the surface of a subject's body, the body signal representing a movement of an object within the subject's body, the position of the Doppler radar acquisition device being associated in a defined way with the position of a sensor for acquiring a vital parameter the subject, and
computer instructions to generate a positioning signal depending on the result of the comparison,

when the computer program is executed in the computer.

This object is also achieved according to the invention by a system and method of generating a target signal and a corresponding system and method of positioning a sensor using said target signal as well as by a computer program, as described below.

The method of generating a target signal for the positioning of a sensor for acquiring a vital parameter of a subject comprises the steps of:
acquiring by means of a Doppler radar acquisition device a body signal representing a movement of an object within the subject's body,
assigning by means of a processing device the acquired body signal to a certain surface position of the subject's body in a way that a virtual map of the surface of the subject's body is generated, said surface position being associated in a defined way with the position of the Doppler radar acquisition device, and
storing by means of a data storage device the resulting entry of the virtual map.

The corresponding method of positioning a sensor for acquiring a vital parameter of a subject comprises the steps of:
selecting by means of a processing device from a virtual map a target body signal according to a desired target sensor position which is associated with a target position of a Doppler radar acquisition device,
during movement of the sensor across the surface of the subject's body, acquiring by means of the Doppler radar acquisition device a current body signal representing a movement of an object within the subject's body, the current position of the sensor being associated in a defined way with the current position of the Doppler radar acquisition device,
comparing by means of a processing device the current body signal with the target body signal, and
generating by means of the processing device a positioning signal depending on the result of the comparison.

The system for generating a target signal for the positioning of a sensor for acquiring a vital parameter of a subject comprises:
a Doppler radar acquisition device adapted to acquire a body signal representing a movement of an object within the subject's body,
a processing device adapted to assign the acquired body signal to a certain surface position of the subject's body in a way that a virtual map of the surface of the subject's body is generated, said surface position being associated in a defined way with the position of the Doppler radar acquisition device, and
a data storage device adapted to store the resulting entry of the virtual map.

The corresponding system for acquiring a vital parameter of a subject by means of a sensor comprises:
a processing device adapted to select from a virtual map a target body signal according to a desired target sensor position which is associated with a target position of a Doppler radar acquisition device,
a Doppler radar acquisition device adapted to acquire during movement of the sensor across the surface of the subject's body a current body signal representing a movement of an object within the subject's body, the current position of the sensor being associated in a defined way with the current position of the Doppler radar acquisition device,
a processing device adapted to compare the current body signal with the target body signal, and further adapted to generate a positioning signal depending on the result of the comparison.

The corresponding computer program to be executed in a computer comprises:
computer instructions to assign a body signal to a certain surface position of a subject's body in a way that a virtual map of the surface of the subject's body is generated, said body signal representing a movement of an object within the subject's body and being acquired by means of a Doppler radar acquisition device, said surface position being associated in a defined way with the position of the Doppler radar acquisition device,
computer instructions to select from a virtual map a target body signal according to a desired target sensor position which is associated with a target position of a Doppler radar acquisition device,
computer instructions to compare a current body signal with a target body signal,

said current body signal representing a movement of an object within the subject's body and being acquired by means of a Doppler radar acquisition device during movement of the sensor across the surface of the subject's body, the current position of the sensor being associated in a defined way with the current position of the Doppler radar acquisition device, and
computer instructions to generate a positioning signal depending on the result of the comparison.

The object of the present invention is also achieved according to the invention by the use of a Doppler radar acquisition device for generating a reference or target signal for the positioning of a sensor for acquiring a vital parameter of a subject, said Doppler radar acquisition device being adapted to acquire a body signal representing a movement of an object within the subject's body. Furthermore the object of the present invention is also achieved according to the invention by the use of a Doppler radar acquisition device for positioning a sensor for acquiring a vital parameter of a subject according to a reference or target signal, said Doppler radar acquisition device being adapted to acquire a body signal representing a movement of an object within the subject's body.

All appliances are adapted to carry out the method according to the present invention. All devices, in particular the processing device, are constructed and programmed in a way that the procedures for obtaining data and for data processing run in accordance with the method of the invention. The technical effects necessary according to the invention can be realized on the basis of the instructions of the computer program in accordance with the invention. Such a computer program can be stored on a carrier such as a CD-ROM or it can be available over the internet or another computer network. Prior to executing the computer program is loaded into the computer by reading the computer program from the carrier, for example by means of a CD-ROM player, or from the internet, and storing it in the memory of the computer. The computer includes inter alia a central processor unit (CPU), a bus system, memory means, e.g. RAM or ROM etc., storage means, e.g. floppy disk or hard disk units etc. and input/output units. Alternatively, the inventive method could be implemented in hardware, e.g. using one or more integrated circuits.

A core idea of the invention is to improve the positioning of a sensor for acquiring a vital parameter of a subject by use of a Doppler radar acquisition device. In the following both “Doppler radar acquisition device” and “Doppler radar sensor” are used.

In such a Doppler radar sensor, as is known in the art, an antenna emits an electromagnetic wave which, when it is reflected from the surfaces of an object moving with a component of velocity non-transverse to the impinging electromagnetic wave, produces a shift in the frequency of the electromagnetic wave reflected back to the antenna. This shift in frequency is called the doppler shift. This doppler shifted reflected wave is detected by an antenna in the sensor, which may or may not be the same antenna as the emitting antenna. The relative speed of movement of the reflecting object is encoded in the frequency shift of the detected reflected wave and this value can be extracted using known techniques. The invention suggests to use a Doppler radar sensor in a way that the emitted electromagnetic signal is used to penetrate the subject's body and to be reflected back from any internal object within the subject's body. The electromagnetic signal becomes doppler shifted in the event that e.g. a reflecting organ is moving relative to the sensor or the electromagnetic signal is reflected back from a passing pulse wave, or any other moving object within the subject's body. This doppler shifted signal is detected by the sensor and can be used to generate a “fingerprint” of the scanned body area depending on the sensor's position. Thus, the resulting signal can be used to generate a virtual map of the subject's body. This virtual map can comprise only one entry, some entries (e.g. 16) or even a very large number of entries (e.g. 100). On the one hand this map can be used for defining the position of a measuring sensor (e.g. an ECG electrode), which already is positioned on the surface of the subject's body. In this case the defined position can be recovered during the next use of the measuring sensor. On the other hand the map can be generated without a sensor being positioned on the surface of the subject's body. In this case the first time positioning of measuring sensors can be guided. Thus, a simple and reliable technique for positioning sensors for acquiring vital parameters of a subject is provided. Furthermore a simple and reliable technique for reproducing the position of sensors is provided.

Preferably the invention can be used for positioning of measuring sensors on a subject's chest. Using Doppler radar the mechanical activity of the heart can be measured. Placing the Doppler radar sensor on the subject's thorax, the radar radiation is reflected at layers of different conductivity. In the thorax major changes in conductivity occur at the heart wall and at the major blood vessels like the aorta. The signal that is measured using Doppler Radar is strongly position dependent, because the reflected signal travels different paths through the thorax depending on the position of the Doppler radar sensor. Every different path a signal takes results in a different signal morphology. Measurements on different positions on the thorax illustrate the different signal morphologies. This essential feature is used according to the invention in a reverse manner to determine the position of the Doppler radar sensor, which can be used to generate the virtual map.

The invention can preferably be used in any application in which the position of sensors on a subjects' body, e.g. the thorax, has a substantial impact on the quality and reproducibility of the measurement. An example is the measurement of ECG, in which the positions of the electrodes have a large impact on the specific measured ECG vectors. Ensuring the measurement of relevant vectors of the ECG will help in better judging of the relevant characteristic features of an ECG and therefore improve diagnosis. Another example is impedance cardiography, in which the position of the electrodes has an impact on the value of Z₀, the non-changing part of the impedance curve, that is a measure for the tissue composition of the thorax. Electrodes positions also have an impact on the actual impedance cardiography signal. Yet another example is the application in an ECG strap, in which case the invention can assure that electrode positions stay the same and are not changed because of different postures or activity of the user. By this means a sufficient signal quality can be ensured. Another example is the positioning of microphones used to record heart sounds. More generally, the invention can be used to ensure a simple and straightforward placement of measuring sensors. Measuring sensors can include all kind of sensors, electrodes etc. for acquiring a vital parameter of a subject.

An automatic support for sensor placement based on the present invention will make it possible to let untrained persons position sensors on medically relevant positions of a subject's body without the need for medically educated persons. Such an automatic support is preferably given by means of visual or acoustic signals based on the positioning signal generated by the processing means of the systems described.

These and other aspects of the invention will be described in detail hereinafter, by way of example, with reference to the following embodiments and the accompanying drawings; in which:

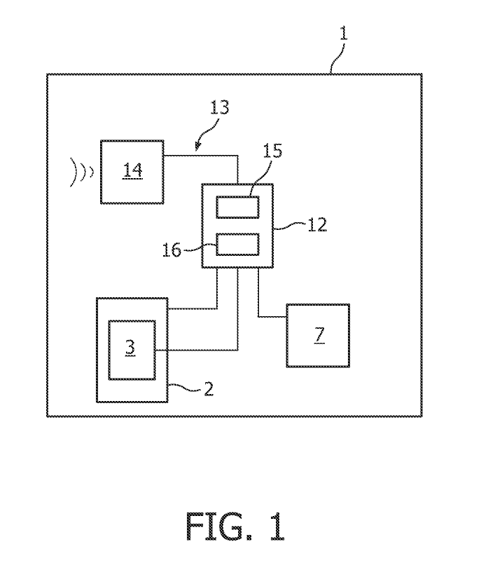

FIG. 1 shows a schematic block diagram of a system according to the invention,

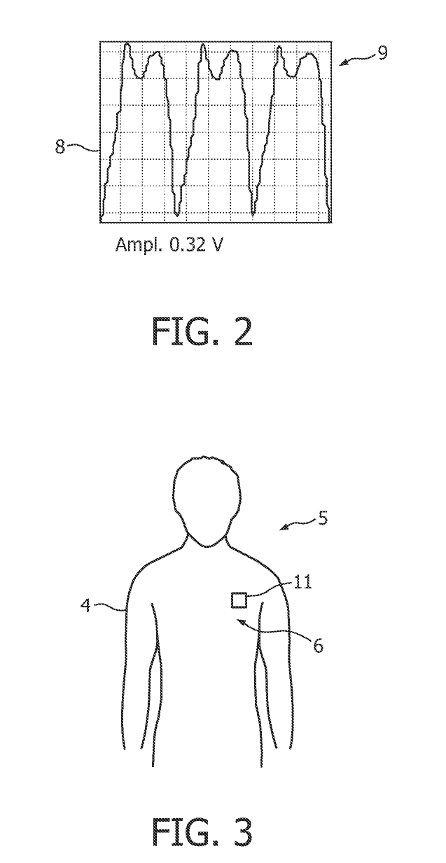

FIG. 2 shows a single body signal,

FIG. 3 shows the surface of a subject's body,

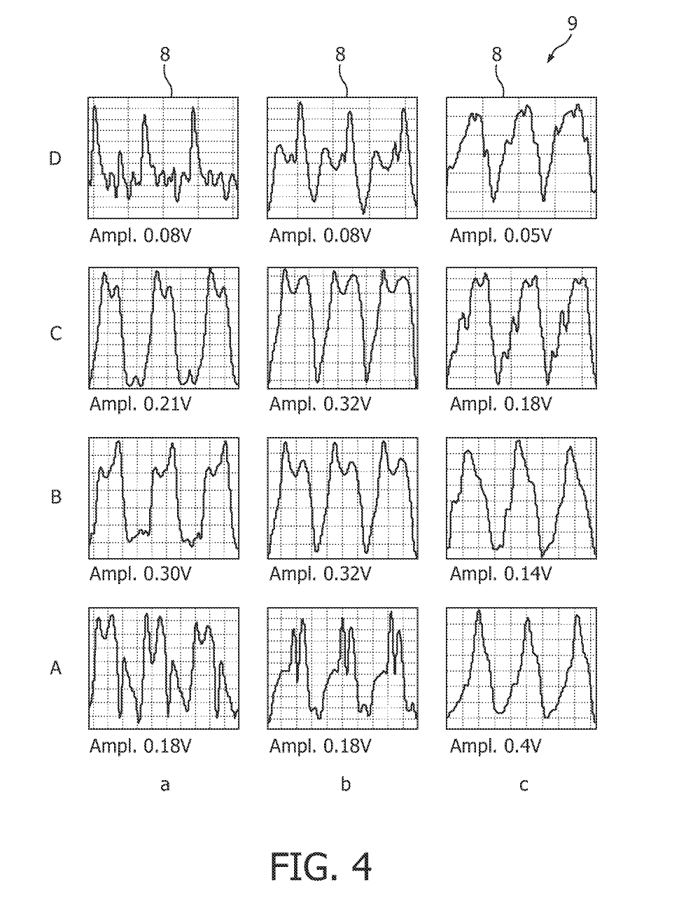

FIG. 4 shows a number of body signals of a virtual map, and

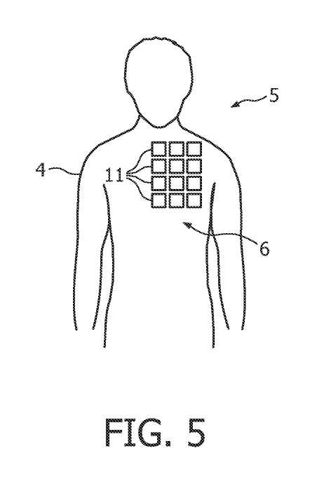

FIG. 5 shows the surface of a subject's body.

In the following embodiments, an ECG electrode is used by way of example as measuring sensor for acquiring vital parameters of a subject. However, the present invention is not limited to ECG electrodes. Other measuring sensors can be used as well, e.g. impedance sensors, microphones etc.

A system 1 according to the present invention for positioning such an ECG electrode 2 is illustrated in FIG. 1. The system 1 comprises a Doppler radar sensor 3. In a first embodiment of the invention the Doppler radar sensor 3 is adapted to acquire a reference body signal S_{R} representing a movement of a heart within a body 4 of a subject 5, see FIG. 3. The Doppler radar sensor 3 comprises a transducer with a 2.45 GHz oscillator. Oscillator and receiver are positioned in the same housing and work in continuous wave mode. However, other frequencies, in particular electromagnetic signals with a frequency in a range of between 400 MHz and 5 GHz, and a pulsed wave mode can be employed.

The position P_{D} of the Doppler radar sensor 3 is associated in a defined way with the position P_{S} of the ECG electrode 2, which is positioned on the surface 6 of the subject's body 4. Preferably the Doppler radar sensor 3 is integrated into to the housing of the ECG electrode 2. Alternatively the Doppler radar sensor 3 is coupled to the ECG electrode 2. The coupling can be implemented in a way that the Doppler radar sensor 3 and the ECG electrode 2 are directly joined together by means of a clamp or any other coupling means. Alternatively the coupling can be implemented in a way that the Doppler radar sensor 3 and the ECG electrode 2 are connected to each other by means of an interfacing element, e.g. a connecting rod. It is important that the position P_{D} of the Doppler radar sensor 3 is associated in a defined and preferably constant way with the position Pₛ of the ECG electrode 2.

Furthermore the system 1 comprises a data storage device 7 adapted to store the acquired reference body signal S_{R}. For example the data storage device 7 may comprise local memory elements, bulk storage, and cache memories, as known in the art. System 1 and data storage device may as well be implemented as separate units, connectable using a wireless communication line.

After the ECG electrode 2 has been positioned on the subject's chest, e.g. by a doctor or nurse, a reference body signal S_{R} representing a movement of the subject's heart is acquired by means of the Doppler radar sensor 3 and the acquired reference body signal S_{R} is stored by means of the data storage device 7. In other words, an entry 8 of a virtual map 9 of the subject's chest is generated. An example of such a virtual map 9 comprising one entry 8 is given in FIG. 2. For the position of the ECG electrode 2 a unique body signal S_{R} is provided by means of the Doppler radar sensor 3. The entry 8 corresponds to a measuring position 11 (surface position P) of the Doppler radar sensor 3 on the subject's chest, as illustrated in FIG. 3.

An entry of the virtual map comprises at least two values, one body signal acquired by means of the Doppler radar sensor and at least one positioning signal. The positioning signal represents the position of the Doppler radar sensor, if the Doppler radar sensor is integrated into to the housing of the ECG electrode, that is if Doppler radar sensor and ECG electrode take the same position relative to the subject. Alternatively a single positioning signal is stored in the case of a coupling means, if there is a known positioning relation between the Doppler radar sensor and the ECG electrode. If the position of the Doppler radar sensor is different to the position of the ECG electrode, two positioning signals are stored into one entry of the virtual map. This is especially the case, if it is necessary that both the position of the Doppler radar sensor and the position of the ECG electrode can be determined separately.

If the ECG electrode 2 shall be positioned again for carrying our the next ECG measurement, the following procedure is suggested: The ECG electrode 2 (together with the Doppler radar sensor 3) is moved across the surface 6 of the subject's chest and a current body signal S_{C} representing a movement of the subject's heart is acquired by means of the Doppler radar sensor 3. The system 1 comprises a processing device 12, by means of which the current body signal S_{C} is compared with the reference body signal S_{R} obtained in the prior step. Depending on the result of the comparison the processing device 12 generates a positioning signal 13, which can be used in a subsequent step, e.g. by the subject 5 to guide the ECG electrode 2 to the correct position, without the help of a doctor or nurse. E.g. an acoustic signal can be provided by means of a signaling device 14, as a result of the positioning signal 13. Preferably the signaling device 14 comprises a speech generator, which is adapted to guide the subject using the words “yes” (i.e. correct position has been found) or “no” (i.e. correct position has not yet been found). In other words, for subsequent measurements the Doppler radar sensor 3 is moved over the subject's chest until the measured body signal S_{C} equals the previously recorded body signal S_{R} at that location. At this position the ECG electrode 2 can be applied and the measurement can be reproduced without any influences due to wrong electrode positioning. Alternatively, if the ECG electrode 2 is not integrated into or coupled to the Doppler radar sensor 3, the correct position of the ECG electrode 2 can be determined first using the Doppler radar sensor 3. In a next step, the found position on the subject's body is automatically marked, e.g. by means of a marking device, e.g. an ink-jet printer device built into the Doppler radar sensor 3. Alternatively the marking is performed by a doctor or nurse or by the subject itself. Finally, the Doppler radar sensor is removed and the ECG electrode 2 is positioned on the marker.

The processing device 12 is adapted for performing all tasks of calculating and computing the received signals and data as well as determining and assessing the results as described. This is achieved according to the invention by means of a computer program 15 comprising computer instructions adapted for carrying out the steps of the inventive method, when the software is executed in the processing device 12. The processing device 12 itself may comprise functional modules or units, which are implemented in form of hardware, software or in form of a combination of both. In particular a computer program 15 to be executed in the processing device 12 is provided, which comprises computer instructions to compare the current body signal S_{C} with the reference body signal S_{R}, and computer instructions to generate a positioning signal depending on the result of the comparison, when the computer program 15 is executed in the processing device 12.

The same system 1 can be used to implement the second embodiment of the invention, as described below. In this embodiment, a virtual map 9 is generated without any ECG electrode 2 being positioned.

The Doppler radar sensor 3 acquires a body signal S representing a movement of the heart within the subject's body. The processing device 12 assigns the acquired body signal S to a certain surface position P of the subject's body 4 in a way that a virtual map 9 of the surface 6 of the subject's body 4 is generated, said surface position P being associated in a defined way with the position P_{D} of the Doppler radar sensor 3. For this assigning step an assigning function is implemented by means of the processing device 12.

In order to generate a virtual map 9 with multiple entries 8, the steps as described above are repeated a number of times for multiple positions of the Doppler radar sensor 3, e.g. signals are recorded for twelve different positions. The resulting entries 8 of the virtual map 9 are stored by means of the data storage device 7. An example of a virtual map 9 comprising twelve 8 entries is given in FIG. 4. The map 9 is formed by a 4×3 matrix with four vertical positions A, B, C, D and three horizontal entries a, b, c for each vertical position. For each position of the Doppler radar sensor 3 a different body signal S is provided. Each body signal S is unique due to it's signal shape (waveform and amplitude). Each entry 8 corresponds to a different measurement positions 11 (surface positions P) on the subject's chest, as illustrated in FIG. 5, where the different measurement positions 11 are shown as a matrix of points on the chest of the subject 5.

If an ECG electrode 2 shall be positioned on the subject's chest, the processing device 12 selects from the virtual map 9 a target body signal S_{T} according to a desired target sensor position P_{T} which is associated with a target position P_{DT} of the Doppler radar sensor 3. The target sensor position P_{T} is known, e.g. by means of a target sensor position map, which is mapped onto the virtual map 9 by means of the processing device 12. In other words, the known target sensor positions P_{T} are virtually combined with the entries 8 of the virtual map 9 in a way that for each target sensor position P_{T} a target body signal S_{T} and a target position P_{DT} of the Doppler radar sensor is known. The target sensor position P_{T} is identical with the target position P_{DT} of the Doppler radar sensor 3, if the Doppler radar sensor 3 is integrated into the ECG electrode, as described above. For positioning the ECG electrode 2 on its target position P_{T} the Doppler radar sensor 3 acquires during movement of the ECG electrode 2 across the subject's chest a current body signal S_{c}. The processing device 12 compares the current body signal S_{C} with the target body signal S_{T} and generates a positioning signal 13 depending on the result of the comparison. In other words, by systematically scanning the subject's chest with the Doppler radar sensor 3, the target sensor position P_{T} for the ECG electrode 3 can be found by comparing the measured body signal S with the target body signal S_{T} on the virtual map 9. In case the exact position for the ECG electrode 3 cannot be restored, i.e. the current Doppler signal does not perfectly coincide with the previously measured Doppler signal, a correlation, e.g. a cross-correlation between stored and measured Doppler signals, can be used to determine, if the reached position can be judged an adequate sensor position for a new measurement. In case of a correlation of e.g. 90% or better, a positioning signal 13 is generated, according to which the current position is adequate. The correlation can be adapted depending on the accuracy required.

Again, the positioning signal 13 can be used by the subject 5 to guide the ECG electrode 2 to the correct position, without the help of a doctor or nurse. E.g. an acoustic signal can be provided by means of the signaling device 14. Preferably the signaling device 14 comprises a speech generator in a way that the subject is guided using the words “yes” and “no” (see above) or “left”, “right”, “up” and “down”, if appropriate signal analyzing is carried out by means of the processing device 12.

For this embodiment in the processing device 12 a computer program 16 is executed, which comprises computer instructions to assign a body signal S to a certain surface position P of a subject's body 4 in a way that a virtual map 9 of the surface 6 of the subject's body 4 is generated, to select from a virtual map 9 a target body signal S_{T} according to a desired target sensor position P_{T} which is associated with a target position P_{DT} of a Doppler radar acquisition device, to compare a current body signal S_{c} with a target body signal S_{T}, and to generate a positioning signal 13 depending on the result of the comparison, when the computer program 16 is executed in the processing device 12.

Preferably the system 1 is adapted in a way that both embodiments described are implemented. In other words, the described system 1 can preferably be used for both generating a reference and a target signal and for positioning a measuring sensor, e.g. an ECG electrode 2, respectively. Accordingly, only one computer program 15, 16 may be provided, comprising computer instructions to implement both methods.

For example the present invention can be used with dry ECG electrodes 2, which are positioned in a belt around the subject's chest. Each ECG electrode 2 is assigned to a Doppler radar sensor 3. If the position of the ECG electrodes 2 changes, the Doppler radar signals recorded by means of the integrated Doppler radar sensors 3 will immediately change its morphology. If such a position change occurs, the generated positioning signal is preferably used to generate a warning signal by means of the signaling device 14, in order to maintain or restore the correct electrode positions. By this means a good quality and reproducible measurement can be ensured. Alternatively an automatic position adjustment system (not shown) can be applied to reposition the ECG electrodes 2. Such an automatic position adjustment system would receive control signals and/or measuring signals from the processing device 12 in order to readjust the electrode's position.

In a further embodiment the ECG electrodes 2 are positioned to each other in an unchangeable way, i.e. the relative positions of the ECG electrodes 2 to each other are constant. In this case only one Doppler radar sensor 3 is used for positioning all ECG electrodes 2 at once.

The positioning of the measuring sensors is not limited to the subject's chest. Measuring sensors (and Doppler radar sensors) can be positioned e.g. on the subject's arms, legs etc. E.g. the movement of passing pulse waves in a subject's leg can be used to generate an entry 8 in the virtual map 9.

It will be evident to those skilled in the art that the invention is not limited to the details of the foregoing illustrative embodiments, and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein. It will furthermore be evident that the word “comprising” does not exclude other elements or steps, that the words “a” or “an” do not exclude a plurality, and that a single element, such as a computer system or another unit may fulfil the functions of several means recited in the claims. Any reference signs in the claims shall not be construed as limiting the claim concerned.

### REFERENCE NUMERALS

1 system
2 ECG electrode
3 Doppler radar sensor
4 body
5 subject
6 surface
7 data storage device
8 entry
9 map
10 (free)
11 measuring device
12 processing device
13 positioning signal
14 signaling device
15 computer program
16 computer program

1. A method of generating a reference signal (S_{R}) for the positioning of a sensor (2) for acquiring a vital parameter of a subject (5), comprising the steps of:
positioning the sensor (2) on the surface (6) of the subject's body (4),
acquiring by means of a Doppler radar acquisition device (3) a reference body signal (S_{R}) representing a movement of an object within the subject's body (4), the position (P_{D}) of the Doppler radar acquisition device (3) being associated in a defined way with the position (Pₛ) of the sensor (2), and
storing the acquired reference body signal (S_{R}) by means of a data storage device (7).

2. A method of positioning a sensor (2) for acquiring a vital parameter of a subject (5), comprising the steps of:
during movement of the sensor (2) across the surface (6) of the subject's body (4), acquiring by means of a Doppler radar acquisition device (3) a current body signal (S_{C}) representing a movement of an object within the subject's body (4), the position (P_{D}) of the Doppler radar acquisition device (3) being associated in a defined way with the position (P_{S}) of the sensor (2),
comparing by means of a processing device (12) the current body signal (S_{C}) with a reference body signal (S_{R}), and
generating by means of the processing device (12) a positioning signal (13) depending on the result of the comparison.

3. A method of generating a target signal (S_{T}) for the positioning of a sensor (2) for acquiring a vital parameter of a subject (5), comprising the steps of:
acquiring by means of a Doppler radar acquisition device (3) a body signal (S) representing a movement of an object within the subject's body (4),
assigning by means of a processing device (12) the acquired body signal (S) to a certain surface position (11, P) of the subject's body (4) in a way that a virtual map (9) of the surface (6) of the subject's body (4) is generated, said surface position (11, P) being associated in a defined way with the position (P_{D}) of the Doppler radar acquisition device (3), and
storing by means of a data storage device (7) the resulting entry of the virtual map (9).

4. A method of positioning a sensor (2) for acquiring a vital parameter of a subject (5), comprising the steps of:
selecting by means of a processing device (12) from a virtual map (9) a target body signal (S_{T}) according to a desired target sensor position (P_{T}) which is associated with a target position (P_{DT}) of a Doppler radar acquisition device (3),
during movement of the sensor (2) across the surface (6) of the subject's body (4), acquiring by means of the Doppler radar acquisition device (3) a current body signal (S_{c}) representing a movement of an object within the subject's body (4), the current position (P_{c}) of the sensor (2) being associated in a defined way with the current position (P_{DQ}) of the Doppler radar acquisition device (3),
comparing by means of a processing device (12) the current body signal (S_{C}) with the target body signal (S_{T}), and
generating by means of the processing device (12) a positioning signal (13) depending on the result of the comparison.

5. A system (1) for generating a reference signal (S_{R}) for the positioning of a sensor (2) for acquiring a vital parameter of a subject (5), comprising:
a Doppler radar acquisition device (3) adapted to acquire a reference body signal (S_{R}) representing a movement of an object within the subject's body (4), the position (P_{D}) of the Doppler radar acquisition device (3) being associated in a defined way with the position (P_{S}) of the sensor (2), which is positioned on the surface (6) of the subject's body (4), and
a data storage device (7) adapted to store the acquired reference body signal (S_{R}).

6. A system (1) for acquiring a vital parameter of a subject (4) by means of a sensor (2), comprising:
a Doppler radar acquisition device (3) adapted to acquire a current body signal (S_{C}) during movement of the sensor (2) across the surface (6) of the subject's body (4), the body signal (S_{C}) representing a movement of an object within the subject's body (4), the position (P_{D}) of the Doppler radar acquisition device (3) being associated in a defined way with the position (Pₛ) of the sensor (2),
a processing device (12) adapted to compare the current body signal (S_{C}) with a reference body signal (S_{R}), and further adapted to generate a positioning signal (13) depending on the result of the comparison.

7. A system (1) for generating a target signal (S_{T}) for the positioning of a sensor (2) for acquiring a vital parameter of a subject (5), comprising:
a Doppler radar acquisition device (3) adapted to acquire a body signal (S) representing a movement of an object within the subject's body (4),
a processing device (12) adapted to assign the acquired body signal (S) to a certain surface position (11, P) of the subject's body (4) in a way that a virtual map (9) of the surface (6) of the subject's body (4) is generated, said surface position (P) being associated in a defined way with the position (P_{D}) of the Doppler radar acquisition device (3), and
a data storage device (7) adapted to store the resulting entry of the virtual map (9).

8. A system (1) for acquiring a vital parameter of a subject (5) by means of a sensor (2), comprising:
a processing device (12) adapted to select from a virtual map (9) a target body signal (S_{T}) according to a desired target sensor position (P_{T}) which is associated with a target position (P_{DT}) of a Doppler radar acquisition device (3),
a Doppler radar acquisition device (3) adapted to acquire during movement of the sensor (2) across the surface (6) of the subject's body (4) a current body signal (S_{C}) representing a movement of an object within the subject's body (4), the current position (P_{C}) of the sensor (2) being associated in a defined way with the current position (P_{DC}) of the Doppler radar acquisition device (3), and
a processing device (12) adapted to compare the current body signal (S_{c}) with the target body signal (S_{T}), and further adapted to generate a positioning signal (13) depending on the result of the comparison.

9. A computer program (15) to be executed in a computer (12), the program comprising:
computer instructions to compare a current body signal (S_{C}) with a reference body signal (S_{R}), said current body signal (S_{c}) being acquired by means of a Doppler radar acquisition device (3) during movement of a sensor (2) across the surface (6) of a subject's body (4), the body signal (S_{c}) representing a movement of an object within the subject's body (4), the position (P_{D}) of the Doppler radar acquisition device (3) being associated in a defined way with the position (P_{S}) of a sensor (2) for acquiring a vital parameter the subject (5), and
computer instructions to generate a positioning signal (13) depending on the result of the comparison,
when the computer program (15) is executed in the computer (12).

10. A computer program (16) to be executed in a computer (12), the program comprising:
computer instructions to assign a body signal (S) to a certain surface position (11, P) of a subject's body (4) in a way that a virtual map (9) of the surface (6) of the subject's body (4) is generated, said body signal (S) representing a movement of an object within the subject's body (4) and being acquired by means of a Doppler radar acquisition device (3), said surface position (11, P) being associated in a defined way with the position (P_{D}) of the Doppler radar acquisition device (3),
computer instructions to select from a virtual map (9) a target body signal (S_{T}) according to a desired target sensor position (P_{T}) which is associated with a target position (P_{DT}) of a Doppler radar acquisition device (3),
computer instructions to compare a current body signal (S_{C}) with a target body signal (S_{T}), said current body signal (S_{c}) representing a movement of an object within the subject's body (4) and being acquired by means of a Doppler radar acquisition device (3) during movement of the sensor (2) across the surface (6) of the subject's body (4), the current position (P_{C}) of the sensor (2) being associated in a defined way with the current position (P_{DQ}) of the Doppler radar acquisition device (3), and
computer instructions to generate a positioning signal (13) depending on the result of the comparison.

11. Use of a Doppler radar acquisition device (3) for generating a reference or target signal (S_{R}, S_{T}) for the positioning of a sensor (2) for acquiring a vital parameter of a subject (5), said Doppler radar acquisition device (3) being adapted to acquire a body signal (S) representing a movement of an object within the subject's body (4).

12. Use of a Doppler radar acquisition device (3) for positioning a sensor (2) for acquiring a vital parameter of a subject (5) according to a reference or target signal (S_{R}, S_{T}), said Doppler radar acquisition device (3) being adapted to acquire a body signal (S) representing a movement of an object within the subject's body (4).
